(19) United States
(12) Patent Application Publication
(10) Pub. No.: US 2009/0283097 A1
(43) Pub. Date: Nov. 19, 2009

(54) MEDICAL CANNULA ASSEMBLY FOR USE WITH A PATIENT

(75) Inventor: Paul J. Niklewski, Cincinnati, OH (US)

Correspondence Address: THOMPSON HINE L.L.P.;Intellectual Property Group, P.O. BOX 8801, DAYTON, OH, 45401-8801, US

(73) Assignee: ETHICON ENDO-SURGERY, INC., Cincinnati, OH (US)

(21) Appl. No.: 12/123,048

(22) Filed: May 19, 2008

(51) Int. Cl. A61M 16/00; A61B 5/083

(52) U.S. Cl. 128/207.14; 600/531

(57) ABSTRACT

A first cannula assembly includes a cannula attachable to a patient to extend along and at least partially cover one side of the patient's face and leave completely exposed the other side of the patient's face. A second cannula assembly includes a reusable unit and a disposable and replaceable loop-shaped mouthpiece. The reusable unit contains a suction pump having a flow inlet and a flow outlet, a power source operatively connected to the suction pump, a carbon dioxide sensor in fluid communication with the flow outlet, and a microprocessor adapted to use at least the carbon dioxide sensor to calculate a carbon dioxide level. The mouthpiece has first and second ends, wherein the first end is directly attached to the reusable unit and the second end is positionable in a patient's mouth. The mouthpiece includes at least one respiratory-gas-sampling port in fluid communication with the flow inlet of the pump.

### FIELD OF THE INVENTION

The present invention is related generally to medical technology, and more particularly to a medical cannula assembly for use with a patient.

### BACKGROUND OF THE INVENTION

Known medical systems having medical cannula assemblies for use with a patient include a cannula which partially covers the nose, the mouth, and both sides of the face of the patient. The cannula includes a cannula body having projecting ports partially covering the nose and mouth and having tubes extending from both sides of the cannula body partially covering both sides of the face. A left-side support wing extends from the left side of the cannula body, partially covers the left side of the face, and has a left wing tip portion with an adhesive pad which is removably attachable to the left side of the face. A right-side support wing extends from the right side of the cannula body, partially covers the right side of the face, and has a right wing tip portion with an adhesive pad which is removably attachable to the right side of the face.

Some of the tubes and the ports are configured to convey respiratory-gas from the nose to a cart-based unit of the cannula assembly. The cart-based unit has a capnometer including a suction pump, a CO2 sensor, a pressure sensor, and a microprocessor. The suction pump brings the nasal respiratory gas to the CO2 sensor and the pressure sensor, wherein an end-tidal carbon dioxide level is calculated by the microprocessor during patient exhalation. A parallel or serial arrangement is provided for respiratory-gas from the mouth. The cart-based unit is hooked up to a supply of oxygen-enriched air (sometimes just called “oxygen”), and some of the tubes and the ports are configured to convey the oxygen-enriched air to the nose and the mouth of the patient. The microprocessor also calculates a patient's respiration rate. The microprocessor is cabled to a monitor which displays the end-tidal carbon dioxide level and the patient's respiration rate. The microprocessor shuts off the flow of oxygen-enriched air during patient exhalation for improved accuracy in calculating the end-tidal carbon dioxide level.

Still, scientists and engineers continue to seek improved medical systems having a medical cannula assembly for use with a patient.

### SUMMARY

A first embodiment of the invention is for cannula assembly including a cannula which is attachable to a patient to extend along and at least partially cover one side of the face of the patient and leave completely exposed the other side of the face of the patient.

A second embodiment of the invention is for a cannula assembly including a reusable unit and a disposable and replaceable loop-shaped mouthpiece. The reusable unit contains a suction pump having a flow inlet and a flow outlet, a power source operatively connected to the suction pump, a carbon dioxide sensor is in fluid communication with the flow outlet, and a microprocessor adapted to use at least the carbon dioxide sensor to calculate a carbon dioxide level. The mouthpiece has first and second ends, wherein the first end is directly attached to the reusable unit and the second end is positionable in the mouth of a patient. The mouthpiece includes at least one respiratory-gas-sampling port in fluid communication with the flow inlet of the suction pump.

Several benefits and advantages are obtained from one or more of the embodiments of the invention. In one example, using a cannula assembly which leaves completely exposed one side of the patient's face allows a medical procedure to be performed on the other side of the patient's face while providing the patient with a cannula system. In the same example, the cannula system can be moved to the other side of the patient's face should the previously covered side of the patient's face also need medical attention. In one example of the second embodiment, having a cannula system with a reusable unit which calculates carbon dioxide level and which is directly connected to a mouthpiece positioned in the patients mouth shortens the respiratory-sampling gas pathway between the patient and the carbon dioxide sensor resulting in a faster response time and a smaller pump size compared to the response times and pump sizes associated with using long prior-art connecting tubing between the patient and the carbon dioxide sensor. In the same or a different example of the second embodiment, the shorter pathway results in a better signal to noise ratio because there is less mixing of the respiratory-sampling gas between the patient and the carbon dioxide sensor.

### BRIEF DESCRIPTION OF THE FIGURES

FIG. 1 is schematic diagram of a first embodiment of a cannula assembly of the invention;

FIG. 2 is an external schematic view of a second embodiment of a cannula assembly of the invention; and

FIG. 3 is an internal schematic view of the cannula assembly of FIG. 2.

### DETAILED DESCRIPTION

Before explaining the embodiments of the invention in detail, it should be noted that each is not limited in its application or use to the details of construction and arrangement of parts, instructions, and steps illustrated in the accompanying drawings and description. The illustrative embodiments of the invention may be implemented or incorporated in other embodiments, variations, and modifications, and may be practiced or carried out in various ways. Furthermore, unless otherwise indicated, the terminology employed herein has been chosen for the purpose of describing the illustrative embodiments of the present invention for the convenience of the reader and are not for the purpose of limiting the invention.

It is further understood that any one or more of the following-described embodiments, implementations, etc. can be combined with any one or more of the other following-described embodiments, implementations, etc.

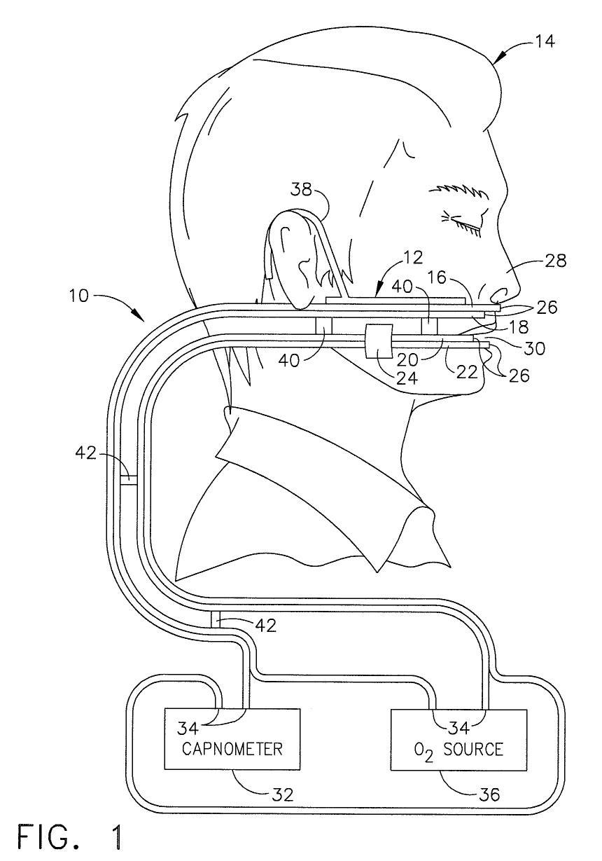

A first embodiment of the invention is shown in FIG. 1 and is for a cannula assembly 10 including a cannula 12 which is attachable to a patient 14 to extend along and at least partially cover one side of the face of the patient 14 and leave completely exposed the other side of the face of the patient 14. In one example, the cannula 12 partially, but not completely, covers the one side of the face of the patient 14.

In one arrangement of the embodiment of FIG. 1, the cannula 12 includes a tube 16, 18, 20 and 22 and an adhesive pad 24. The tube 16, 18, 20 and 22 has a distal port 26. The adhesive pad 24 is adapted to (directly or indirectly) attach the tube 20 and 22 to the one side of the face with the distal port 26 positioned proximate at least one of the nose 28 and the mouth 30 of the patient 14. In one variation, the cannula assembly 10 also includes a capnometer 32 (which measures the carbon dioxide level of the patient's exhaled air), wherein the tube 22 has a proximal port 34 operatively connectable to the capnometer 32. In one modification, the cannula assembly 10 also includes a source of oxygen-enriched air 36, wherein the tube 20 has a proximal port 34 operatively connectable to the source of oxygen-enriched air 36. It is noted that oxygen-enriched air is sometimes referred to simply as oxygen. In one example, the adhesive pad 24 provides the only attachment of the cannula 12 to the patient 14.

In the same or a different arrangement, the cannula 12 includes a tube 16, 18, 20 and 22 and an ear strap 38. The tube 16, 18, 20 and 22 has a distal port 26. The ear strap 38 is adapted to (directly or indirectly) attach the tube 16 and 18 to an ear of the patient 14 with the distal port 26 positioned proximate at least one of the nose 28 and the mouth 30 of the patient 14. In one variation, the cannula assembly 10 also includes a capnometer 32, wherein the tube 16 has a proximal port 34 operatively connectable to the capnometer 32. In one modification, the cannula assembly 10 also includes a source of oxygen-enriched air 36, wherein the tube 18 has a proximal port 34 operatively connectable to the source of oxygen-enriched air 36. In one example, the ear strap 38 provides the only attachment of the cannula 12 to the patient 14. Other arrangements are left to the artisan including those having separate nasal and oral capnometers, those measuring only nasal CO2 (carbon dioxide), those measuring only oral CO2, those delivering only nasal O2 (oxygen), those delivering only oral O2, those with O2 delivery but without CO2 measurement, those with CO2 measurement but without O2 delivery, etc.

In one implementation of the embodiment of FIG. 1, the cannula 12 includes a tube 16 and 18 having a distal port 26 which is positioned proximate the nose 28 of the patient 14 when the cannula 12 is attached to the patient 14. In the same or a different implementation, the cannula 12 includes a tube 20 and 22 having a distal port 26 which is positioned proximate the mouth 30 of the patient 14 when the cannula 12 is attached to the patient 14. Other implementations are left to the artisan.

In one construction of the embodiment of FIG. 1, the cannula assembly 10 includes one or more adhesive pads and does not include an ear strap. In one variation, the adhesive pads are adapted in number and/or in design to allow the cannula 12 to be removably attached to either side of the face of the patient 12. In one example, the adhesive pad or pads are disposed between at least one tube and the one side of the face of the patient. In one illustration, the adhesive pad or pads are directly attached to all of the tubes.

In another construction, the cannula assembly 10 includes an ear strap but does not include an adhesive pad. In one variation, the ear strap is adapted in design to allow the cannula 12 to be removably attached to either ear of the patient 12. In either or a different construction, the cannula assembly 10 includes at least one flexible strap 40 for interconnecting tubes 16 and 18 with tubes 20 and 22 proximate the one side of the face of the patient 14. In one variation, the cannula assembly 10 includes at least one tie 42 interconnecting tubes 16 and 18 with tubes 20 and 22 proximal the at-least-one flexible strap 40 for ease of tube handling. In one modification, tubes 16 and 18 are attached together, side-by-side, substantially along their entire lengths, and tubes 20 and 22 are attached together, side-by-side, substantially along their entire lengths.

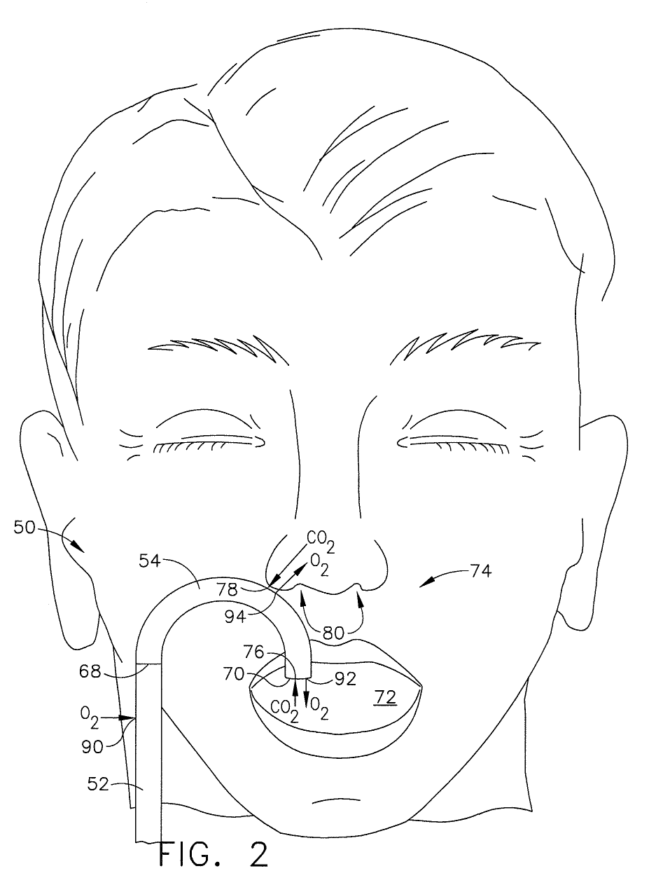

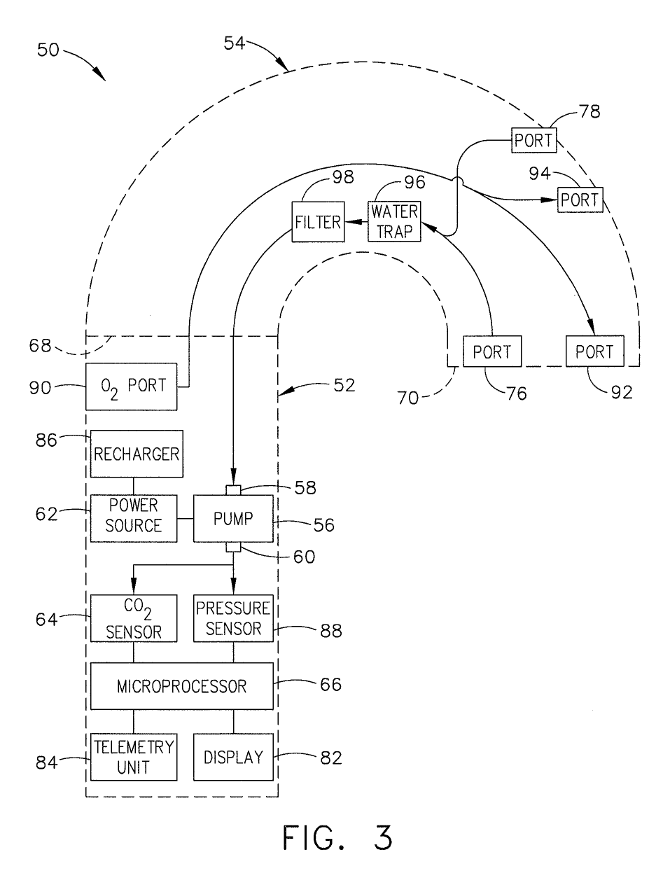

A second embodiment of the invention is shown in FIGS. 2-3 and is for a cannula assembly 50 including, a reusable unit 52 and a disposable and replaceable loop-shaped mouthpiece 54. The reusable unit 52 contains a suction pump 56 having a flow inlet 58 and a flow outlet 60, a power source 62 operatively connected to the pump 56, a carbon dioxide sensor 64 in fluid communication with the flow outlet 60, and a microprocessor 66 adapted to use at least the carbon dioxide sensor 64 to calculate a carbon dioxide level. It is noted that the reusable unit 52, as described in the previous sentence, defines a basic capnometer. The mouthpiece 54 has first and second ends 68 and 70, wherein the first end 68 is directly attached to the reusable unit 52 and the second end 70 is positionable in the mouth 72 of a patient 74. The mouthpiece 54 includes at least one respiratory-gas-sampling port 76 and 78 in fluid communication with the flow inlet 58 of the suction pump 56.

In one example, the mouthpiece 54 with the attached reusable unit 52 will hang from the mouth 72 of a supine, upward-facing patient 74 when the second end 70 is positioned in the mouth 72 of the patient 74. In the same or a different example, the mouthpiece 54 with the attached reusable unit 52 will hang from the mouth 72 of a standing, forward-facing patient 74 when the second end 70 is positioned in the mouth 72 of the patient 74.

In one enablement of the embodiment of FIGS. 2-3, the at-least-one respiratory-gas sampling port 76 and 78 includes a respiratory-gas-sampling port 76 which is positioned in the mouth 72 of the patient 74 when the second end 70 of the mouthpiece 54 is positioned in the mouth 72 of the patient 74. In the same or a different enablement, the at-least-one respiratory-gas sampling port 76 and 78 includes a respiratory-gas-sampling port 78 which is positioned outside the mouth 72 and proximate a nostril 80 of the patient 74 when the second end 70 of the mouthpiece 54 is positioned in the mouth 72 of the patient 74.

In one employment of the embodiment of FIGS. 2-3, the reusable unit 52 includes a display 82 adapted to show the calculated carbon dioxide level. In the same or a different employment, the reusable unit 52 includes telemetry 84 to transmit the calculated carbon dioxide level. In the same or a different employment, the reusable unit 52 includes a recharger 86 adapted to recharge the power source 62. In one variation, the power source is a battery and the recharger is a battery recharger.

In one arrangement of the embodiment of FIGS. 2-3, the mouthpiece 54 has a substantially inverted-“U” shape. In the same or a different arrangement, the mouthpiece 54 is articulatable. An articulatable mouthpiece allows a user to better conform the shape of the loop of the loop-shaped mouthpiece to the particular mouth-nostril geometry of a particular patient so that a nostril port (e.g., respiratory-gas-sampling port 78), when present in the mouthpiece, will be better placed in relation to a nostril of the patient.

In one application of the embodiment of FIGS. 2-3, the reusable unit 52 includes a pressure sensor 88 in fluid communication with to the flow outlet 60, wherein the microprocessor 66 is adapted to use at least the pressure sensor 88 o determine if the patient 74 is inhaling or exhaling and to determine the respiratory rate of the patient 74, and wherein the calculated carbon dioxide level is an end-tidal carbon dioxide level. In one extension of the embodiment of FIGS. 2-3, the reusable unit 52 includes an inlet port 90 adapted to receive oxygen-enriched air (sometimes just called “oxygen”), and the mouthpiece 54 includes at least one respiratory-gas-supply port 92 and 94 in fluid communication with the inlet port 90. It is noted that unlabeled arrows in FIG. 2 indicate gas flow passages inside the mouthpiece 54 and the resuable unit 52 and the reusable unit 52, and that unlabeled lines between components inside the reusable unit 52 indicate interconnections between such components.

In one construction of the embodiment of FIGS. 2-3, the mouthpiece 54 includes a water trap 96. In the same or a different construction, the mouthpiece 54 includes a hydrophobic filter 98.

The presence and arrangements of components different from those shown in FIG. 3 are left to the artisan including those having separate nasal and oral capnometers, those measuring only nasal CO2 (carbon dioxide), those measuring only oral CO2, those delivering only nasal O2 (oxygen), those delivering only oral O2, those with CO2 measurement but without O2 delivery, etc.

Several benefits and advantages are obtained from one or more of the embodiments of the invention. In one example, using a cannula assembly which leaves completely exposed one side of the patient's face allows a medical procedure to be performed on the other side of the patient's face while providing the patient with a cannula system. In the same example, the cannula system can be moved to the other side of the patient's face should the previously covered side of the patient's face also need medical attention. In one example of the second embodiment, having a cannula system with a reusable unit which calculates carbon dioxide level and which is directly connected to a mouthpiece positioned in the patient's mouth shortens the respiratory-sampling gas pathway between the patient and the carbon dioxide sensor resulting in a faster response time and a smaller pump size compared to the response times and pump sizes associated with using long prior-art connecting tubing between the patient and the carbon dioxide sensor. In the same or a different example of the second embodiment, the shorter pathway results in a better signal to noise ratio because there is less mixing of the respiratory-sampling gas between the patient and the carbon dioxide sensor.

While the present invention has been illustrated by several embodiments, and enablements, applications, etc. thereof, it is not the intention of the applicant to restrict or limit the spirit and scope of the appended claims to such detail. Numerous other variations, changes, and substitutions will occur to those skilled in the art without departing from the scope of the invention. It will be understood that the foregoing description is provided by way of example, and that other modifications may occur to those skilled in the art without departing from the scope and spirit of the appended Claims.

What is claimed is:

1. A cannula assembly comprising a cannula which is attachable to a patient to extend along and at least partially cover one side of the face of the patient and leave completely exposed the other side of the face of the patient.

2. The cannula assembly of claim 1, wherein the cannula includes a tube and an adhesive pad, wherein the tube has a distal port, and wherein the adhesive pad is adapted to attach the tube to the one side of the face with the distal port positioned proximate at least one of the nose and the mouth of the patient.

3. The cannula assembly of claim 2, also including a capnometer, wherein the tube has a proximal port operatively connectable to the capnometer.

4. The cannula assembly of claim 2, also including a source of oxygen-enriched air, wherein the tube has a proximal port operatively connectable to the source of oxygen-enriched air.

5. The cannula assembly of claim 1, wherein the cannula includes a tube and an ear strap, wherein the tube has a distal port, and wherein the ear strap is adapted to attach the tube to an ear of the patient with the distal port positioned proximate at least one of the nose and the mouth of the patient.

6. The cannula assembly of claim 5, also including a capnometer, wherein the tube has a proximal port operatively connectable to the capnometer.

7. The cannula assembly of claim 5, also including a source of oxygen-enriched air, wherein the tube has a proximal port operatively connectable to the source of oxygen-enriched air.

8. The cannula assembly of claim 1, wherein the cannula includes a tube having a distal port which is positioned proximate the nose of the patient when the cannula is attached to the patient.

9. The cannula assembly of claim 8, wherein the cannula includes a tube having a distal port which is positioned proximate the mouth of the patient when the cannula is attached to the patient.

10. A cannula assembly comprising:
a) a reusable unit containing a suction pump having a flow inlet and a flow outlet, a power source operatively connected to the suction pump, a carbon dioxide sensor in fluid communication with the flow outlet, and a microprocessor adapted to use at least the carbon dioxide sensor to calculate a carbon dioxide level; and
b) a disposable and replaceable loop-shaped mouthpiece having first and second ends, wherein the first end is directly attached to the reusable unit and the second end is positionable inside the mouth of a patient; wherein the mouthpiece includes at least one respiratory-gas-sampling port in fluid communication with the flow inlet of the suction pump.

11. The cannula assembly of claim 10, wherein the at-least-one respiratory-gas-sampling port includes a respiratory-gas-sampling port which is positioned in the mouth of the patient when the second end of the mouthpiece is positioned in the mouth of the patient.

12. The cannula assembly of claim 10, wherein the at-least-one respiratory-gas-sampling port includes a respiratory-gas-sampling port which is positioned outside the mouth and proximate a nostril of the patient when the second end of the mouthpiece is positioned in the mouth.

13. The cannula assembly of claim 10, wherein the reusable unit includes a display adapted to show the calculated carbon dioxide level.

14. The cannula assembly of claim 10, wherein the reusable unit includes telemetry to transmit the calculated carbon dioxide level.

15. The cannula assembly of claim 10, wherein the reusable unit includes a recharger adapted to recharge the power source.

16. The cannula assembly of claim 10, wherein the mouthpiece has a substantially inverted-“U” shape.

17. The cannula assembly of claim 10, wherein the mouthpiece is articulatable.

18. The cannula assembly of claim 10, wherein the reusable unit includes a pressure sensor in fluid communication with the flow outlet, wherein the microprocessor is adapted to use at least the pressure sensor to determine if the patient is inhaling or exhaling and to determine the respiratory rate of the patient, and wherein the calculated carbon dioxide level is an end-tidal carbon dioxide level.

19. The cannula assembly of claim 10, wherein the mouthpiece includes at least one of a hydrophobic filter and a water trap.

20. The cannula assembly of claim 10, wherein the reusable unit includes an inlet port adapted to receive oxygen-enriched air, and wherein the mouthpiece includes a respiratory-gas-supply port in fluid communication with the inlet port.
